(19) United States
(12) Patent Application Publication
(10) Pub. No.: US 2021/0393607 A1
(43) Pub. Date: Dec. 23, 2021

(54) Transdermal Patch Containing Fentanyl as Active Ingredient

(71) Applicant: MEDRX CO., LTD., Kagawa (JP)

(72) Inventors: Katsuhiro Yamanaka, Kagawa (JP); Hidetoshi Hamamoto, Kagawa (JP); Yasushi Miwa, Kagawa (JP)

(73) Assignee: MEDRX CO., LTD., Kagawa (JP)

(21) Appl. No.: 17/292,318

(22) PCT Filed: Nov. 7, 2019
(86) PCT No.: PCT/JP2019/043585

§ 371 Date: May 7, 2021

(30) Foreign Application Priority Data
Nov. 9, 2018 (JP) 2018-211327

(51) Int. Cl. A61K 31/4468; A61K 9/70; A61K 47/06; A61K 47/14; A61K 47/10

(52) U.S. Cl. CPC A61K 31/4468; A61K 9/7084; A61K 47/10; A61K 47/14; A61K 47/06

(57) ABSTRACT

Disclosed is a patch containing a plaster having fentanyl, an adhesive, a volatile hydrocarbon solvent (A), and a heteroatom-containing volatile organic solvent (B).

### CROSS-REFERENCE TO RELATED APPLICATIONS

This application claims the benefit of Japanese Patent Application No. 2018-211327, filed on Nov. 9, 2018 with Japanese Patent Office. The Japanese application is hereby incorporated by reference for all purposes as if the entirety application documents (specification, claims, drawings, and abstract) were expressly set forth herein. The present invention belongs to the technical field of patches. The present invention relates to a patch containing fentanyl as an active ingredient, which cannot be reapplied as well as being excellent in sustained release property.

### TECHNICAL FIELD

### Background of the Invention

Nowadays, accidents caused by accidental use of patches are increasing. Traditionally, drugs contained in patches have been topical ones and are generally anti-inflammatory drugs with relatively low physiological activity. However, the use of systemic and/or highly physiologically active drugs as active ingredients in patches is also increasing. The misuse of such a patch can cause severe poisoning. Even after being used according to a prescribed dosage regimen, a patch may contain a certain amount of drugs remaining. The misuse of such patches may cause poisoning accidents. Specifically, in infants, the use of a patch used by an adult may cause a serious accident. Therefore, there has been a desire for a patch coping with the risk of an accident due to such misuse. In order to cope with such a problem, patches having accidental use/abuse prevention properties have been proposed. For example, Patent Documents 1 and 2 propose a method of using a patch device in which a drug such as fentanyl and a binding agent which inactivates the drug are mixed at the time of removal from the skin so as for the device not to be reusable.

Moreover, Patent Document 3 proposes a patch having misuse prevention properties. In Patent Document 3, a volatile solvent having the vapor pressure of 1 kPa or more at 20° C. is contained in a plaster, and when the patch is used, as the volatile solvent evaporates, the adhesive force of the plaster is lost, thereby preventing reapplication of patches. Furthermore, Patent Document 3 also describes a patch containing fentanyl (see paragraphs 0048 to 0051 of Patent Document 3).

### PRIOR ART DOCUMENTS

### Patent Document

Patent Document 1: JP-A-2007-58819
Patent Document 2: JP-A-2010-248238
Patent Document 3: International Publication No. 2017/164084

### SUMMARY OF THE INVENTION

### Problem to be Solved by the Invention

The patch described in Patent Document 3 is excellent in preventing misuse, because it is difficult to reapply the patch if once the release film is removed and the patch is applied to the skin. On the other hand, although the patch of Patent Document 3 is not basically limited to the kinds of drugs and then it is versatile, it is not always satisfactory for individual drugs. Specifically, absorbency of drugs through the skin is not considered.

The present invention is primarily directed to provide a novel patch particularly suitable for fentanyl, which, in addition to having an ability to prevent reapplication, can be used for a long time and which is excellent in adjusting the pharmacokinetic profile, and therefore is advantageous for adjusting the sustained release property.

### Means for Solving the Problems

As a result of intensive studies, the present inventors have found that the above problem can be solved by containing a plurality of volatile organic solvents in a plaster and devising the content ratio thereof, and the present invention was completed.

The present invention can include, for example, the following embodiments.

[1] A patch containing fentanyl as an active ingredient, comprising a plaster having fentanyl, an adhesive, a volatile hydrocarbon solvent (A), and a heteroatom-containing volatile organic solvent (B), wherein the boiling point of the solvent (A) is higher than that of the solvent (B), and the content of the solvent (A) in the plaster is larger than that of the solvent (B) in the plaster.
[2] The patch according to [1] above, wherein the boiling point of the solvent (B) is lower than that of the solvent (A) by 8° C. or more.
[3] The patch according to [1] or [2] above, wherein the ratio of the solvent (B) content in the plaster to the solvent (A) content in the plaster is within the range of 1:1.1 to 1:20 (B:A) by weight.
[4] The patch according to any one of [1] to [3] above, wherein the boiling point of the solvent (A) is within the range of 75 to 140° C.
[5] The patch according to any one of [1] to [4] above, wherein the content of the solvent (B) in the plaster is within the range of 1 to 30 wt %.
[6] The patch according to any one of [1] to [5] above, wherein the solvent (B) is one or more solvents selected from the group consisting of an ester-based solvent, an ether-based solvent, an alcohol-based solvent, an amine-based solvent, a ketone-based solvent, an amide-based solvent, and a chloride-based solvent.
[7] The patch according to any one of [1] to [6] above, wherein the solvent (B) is one or more solvents selected from the group consisting of ethyl acetate, ethanol, and isopropanol.
[8] The patch according to any one of [1] to [7] above, wherein the solvent (A) is one or more solvents selected from the group consisting of n-heptane, toluene, cyclohexane, and n-octane.
[9] The patch according to any one of [1] to [8] above, wherein the adhesive is a mixture of an acrylic polymer and a styrene-isoprene-styrene copolymer.
[10] The patch according to [9] above, wherein the weight ratio of the acrylic polymer to the styrene-isoprene-styrene copolymer in the mixture is within the range of 5:1 to 1:5.
[11] The patch according to any one of [1] to [10] above, further comprising a bitterant.
[12] The patch according to any one of the [1] to [11] above, further comprising a C₈₋₂₂ higher alcohol.
[13] A patch containing fentanyl as an active ingredient,
comprising a drug layer consisting of a plaster having fentanyl, an adhesive, a volatile hydrocarbon solvent (A), and a heteroatom-containing volatile organic solvent (B), and a support; and a cover layer covering and larger than the drug layer, wherein the cover layer comprises an adhesive layer; and wherein the boiling point of the solvent (A) is higher than that of the solvent (B) and the content of the solvent (A) in the plaster is larger than that of the solvent (B) in the plaster.
[14] The patch according to [13] above, wherein the adhesive layer in the cover layer contains a volatile organic solvent.
[15] The patch according to [14] above, wherein the volatile organic solvent is a volatile hydrocarbon solvent (C), a heteroatom-containing volatile organic solvent (D), or a mixed solvent thereof.
[16] The patch according to [15] above, wherein the solvent (C) is an acyclic or cyclic aliphatic hydrocarbon solvent, and the solvent (D) is one or more solvents selected from the group consisting of an ester-based solvent, an ether-based solvent, an alcohol-based solvent, an amine-based solvent, a ketone-based solvent, an amide-based solvent, and a chloride-based solvent.
[17] The patch according to [15] or [16] above, wherein the solvent (C) is one or more solvents selected from the group consisting of n-heptane, toluene, cyclohexane, and n-octane, and the solvent (D) is one or more solvents selected from the group consisting of ethyl acetate, ethanol, and isopropanol.
[18] The patch according to any one of [15] to [17] above, wherein the solvent (C) is a n-heptane and the solvent (D) is ethyl acetate.
[19] The patch according to any one of [12] to [18] above, wherein the C₈₋₂₂ higher alcohol is stearyl alcohol.

### Effect of the Invention

According to the present invention, it is possible to suppress a patch from being reapplied after use and effectively prevent drug abuse and accidents. In addition, it is possible to provide a patch which is advantageous in the absorption of fentanyl from the skin and the adjustment of the pharmacokinetic profile, and which can be used for a long time with excellent sustained release properties of fentanyl.

### BRIEF DESCRIPTION OF THE DRAWINGS

FIG. 1 represents a cross-sectional view of a patch according to the present invention, which have a laminated structure. 1 indicates a plaster having fentanyl, 2 indicates a support which is a nonwoven fabric laminated film, 3 indicates an adhesive layer in a cover layer, and 4 indicates a support which is a nonwoven fabric laminated film. Furthermore, 5 indicates a nonwoven fabric moiety of the support, 6 indicates a film moiety thereof, 7 indicates a film moiety of the support, and 8 indicates a nonwoven fabric moiety thereof.

FIG. 2 represents the amount of skin permeation of fentanyl. The vertical axis indicates the amount of release (μg/cm²), and the horizontal axis indicates the time of release (hours), respectively. In this figure, the black rhombuses (upper curve) indicate the result of the present invention patch of Example 5, and the black squares (lower curve) indicate the result of a commercial product, respectively.

FIG. 3 represents the amount of skin permeation of fentanyl. The vertical axis indicates the amount of release (μg/cm²), and the horizontal axis indicates the time of release (hours), respectively. In this figure, black circles (upper curve) indicate the result of a commercial product, and black rhombuses (lower curve) indicate the result of the present invention patch of Example 5, respectively.

### EMBODIMENT FOR CARRYING OUT THE PRESENT INVENTION

### 1 Patch According to the Present Invention

The patch according to the present invention (hereinafter, referred to as “the present invention patch”) is a patch containing fentanyl as an active ingredient, comprising a plaster having fentanyl, an adhesive, a volatile hydrocarbon solvent (A), and a heteroatom-containing volatile organic solvent (B), wherein the boiling point of the solvent (A) is higher than that of the solvent (B), and the content of the solvent (A) in the plaster is larger than that of the solvent (B) in the plaster.

Hereinafter, the present invention patch will be described in detail.

### 1.1 Plaster

The plaster is usually a thin layer containing a drug and an adhesive, and in the present invention, it has fentanyl, an adhesive, a volatile hydrocarbon solvent (A), and a heteroatom-containing volatile organic solvent (B).

### 1.1.1 Fentanyl

Fentanyl, in the present invention, is a known opioid compound which has a chemical name: N-(1-phenethylpiperidin-4-yl)-N-phenylpropanamide, and is mainly used for anesthesia, analgesia, and pain relief. Manufacturing methods of fentanyl are also known and it can also be obtained from the market.

The content of fentanyl in the plaster is usually within the range of 0.5 to 20 wt % relative to the total weight of the plaster components. Preferably, it is within the range of 1 to 10 wt %. More preferably, it is within the range of 1.2 to 8 wt %. Furthermore preferably, it is within the range of 1.5 to 7 wt %. The content of fentanyl in the plaster may be within the range of 1.7 to 2.5 wt %.

### 1.1.2 Adhesive

The adhesive in the present invention is usually non-aqueous and may comprise a base and optionally a tackifier. The content of the adhesive is usually within the range of 20 to 85 wt % relative to the total weight of the plaster components, preferably within the range of 30 to 80 wt %, more preferably within the range of 40 to 75 wt %, and further more preferably within the range of 50 to 70 wt %.

The base contained in the adhesive may be composed of a polymer. As such a polymer, a rubber-based polymer, an acrylic polymer, a silicone-based polymer, and the like can be exemplified.

Specific examples of the rubber-based polymer can include synthetic rubbers such as styrene-isoprene-styrene block copolymer (hereinafter, sometimes referred to as “SIS”), styrene-butadiene-styrene block copolymer, styrene-ethylene-butadiene rubber-styrene block copolymer, styrene-butadiene rubber, polyisoprene, polyisobutylene, polybutene, and butyl rubber; and natural rubbers.

Specific examples of the acrylic polymer can include polymers or copolymers of acrylic esters such as polyacrylic acid methyl ester and polymethacrylic acid methyl ester, and examples thereof can include DURO-TAK 87-900A, 87-9301, 87-4098, 387-2051, 87-2051, 387-2287, 87-2287, 87-4287, GELVA GMS 3083, 3253 (trade name, manufactured by Henkel Japan Ltd.), and Nissetsu KP-77, AS-370 (trade name, manufactured by Nippon Carbide Industries Co., Inc.).

Specific examples of the silicone-based polymer can include dimethylpolysiloxane and diphenylpolysiloxane.

The above-mentioned base may be used singly or in any combination of two or more kinds.

The base in the present invention preferably comprises an acrylic polymer. The acrylic polymer can achieve both high adhesiveness and an ability to prevent reapplication.

The base in the present invention preferably further comprises a rubber-based polymer. That is, a combination of an acrylic polymer and a rubber-based polymer (SIS or the like) is preferred. The weight ratio of the acrylic polymer to the rubber-based polymer (SIS or the like) is suitably within the range of 5:1 to 1:5, preferably within the range of 3:1 to 1:3, and more preferably within the range of 2:1 to 1:3. The same applies to the mixture of the acrylic polymer and the SIS.

As the tackifier, an alicyclic hydrocarbon resin, a terpene resin, an aliphatic hydrocarbon resin, a polystyrene-based resin, a Rosin, a hydrogenated Rosin, and the like can be exemplified. The tackifier is preferably a terpene resin. The content of the tackifier is usually within the range of 2 to 40 wt %, preferably within the range of 5 to 30 wt %, and more preferably within the range of 8 to 20 wt % relative to the total weight of the adhesive components. The content of the tackifier may be within the range of 13 to 20 wt % relative to the total weight of the adhesive components.

### 1.1.3 Volatile Hydrocarbon Solvent (A) and Heteroatom-Containing Volatile Organic Solvent (B)

A volatile hydrocarbon solvent (A) according to the present invention (hereinafter, simply referred to as “the solvent (A)”) is not particularly limited as long as it is a hydrocarbon solvent having the vapor pressure of 0.1 kPa or more at 20° C. and the boiling point higher than room temperature (25° C.), and is preferably a hydrocarbon solvent having the vapor pressure of 1 kPa or more, or 1 to 20 kPa and the boiling point within the range of 75° C. to 140° C., preferably having the boiling point within the range of 85° C. to 125° C., and more preferably having the boiling point within the range of 90° C. to 110° C.

As the solvent (A), an acyclic or cyclic aliphatic hydrocarbon solvent is preferred and the acyclic aliphatic hydrocarbon solvent is more preferred, and among them, a chain aliphatic hydrocarbon solvent is preferred and a linear aliphatic hydrocarbon solvent is more preferred.

The number of carbon atoms of the solvent (A) is preferably within the range of 4 to 9, more preferably within the range of 5 to 8, further more preferably within the range of 6 to 8, particularly preferably 7 or 8, and most preferably 7.

Specific examples of the solvent (A) can include acyclic or cyclic hydrocarbon solvents such as n-hexane, n-heptane, n-octane, toluene, benzene, cyclohexane, and xylene. Among them, n-heptane is preferred. These solvents (A) may be used singly or in any combination of two or more kinds.

In the present invention, by increasing or decreasing the content of the solvent (A), the sustained release of fentanyl can be controlled.

A heteroatom-containing volatile organic solvent (B) (hereinafter, simply referred to as “the solvent (B)”) is not particularly limited as long as it is a heteroatom-containing volatile organic solvent having the vapor pressure of 0.1 kPa or more at 20° C. and the boiling point higher than room temperature (25° C.), and is preferably a heteroatom-containing volatile organic solvent having the vapor pressure of 1 kPa or more, or 1 to 50 kPa and the boiling point within the range of 65° C. to 100° C., preferably having the boiling point within the range of 75° C. to 85° C., and more preferably having the boiling point within the range of 75° C. to 80° C. Moreover, it is preferable to select a solvent (B) which has a boiling point within the range described above and lower than the boiling point of the solvent (A) by 8° C. or more, or alternatively by 10° C. or more.

The heteroatom contained in the solvent (B) can include, for example, an oxygen atom, a nitrogen atom, a chlorine atom, and a sulfur atom, and among them, an oxygen atom is preferred. Specific examples of the solvent (B) containing an oxygen atom can include ester-based solvents such as ethyl acetate, isobutyl acetate, and isopropyl acetate, which are acetate ester solvents; and formic acid esters; ether-based solvents such as ethyl ether, tetrahydro furan, 1,4-dioxane; alcohol-based solvents such as ethanol, isopropanol, isobutyl alcohol, and 2-butanol; ketone-based solvents such as acetone, methyl ethyl ketone, and methyl isobutyl ketone. Among them, the ester-based solvents and the alcohol-based solvents are preferred, and the ester-based solvents are more preferred. Among the ester-based solvents, acetate ester solvents are preferred, and ethyl acetate is particularly preferred.

Specific examples of the solvent (B) containing a nitrogen atom can include amine-based solvents.

Specific examples of the solvent (B) containing a chlorine atom can include chloride-based solvents.

These solvents (B) may be used singly or in any combination of two or more kinds.

The solvent (A) and the solvent (B) may arbitrarily be combined, and the combination of an acyclic or a cyclic aliphatic hydrocarbon solvent, and an ester-based solvent is preferred. Among them, a combination of the acyclic aliphatic hydrocarbon solvent and the ester-based solvent is preferred, and a combination of a chain aliphatic hydrocarbon solvent and an ester-based solvent is more preferred. Specifically, the combination of n-heptane and ethyl acetate is preferred.

In the present invention, the content of the solvent (A) in the plaster is larger than that of the solvent (B) in the plaster. The content ratio thereof varies depending on the desired sustained release property and the like, but is usually within the range of 1:1.1 to 1:20 (B:A), preferably within the range of 1:1.2 to 1:10 (B:A), more preferably within the range of 1:1.3 to 1:5 (B:A), and further more preferably within the range of 1:1.5 to 1:3 (B:A) by weight. The content ratio thereof may be within the range of 1:2 to 1:3 (B:A), or 1:2.5 to 1:3 (B:A).

The content of the solvent (B) in the plaster is usually within the range of 1 to 30 wt % relative to the total weight of the plaster components, preferably within the range of 2 to 25 wt %, more preferably within the range of 5 to 20 wt %, and further more preferably within the range of 5 to 10 wt %.

The combined content of the solvent (A) and the solvent (B) is usually in the range of 5 to 45 wt %, relative to the total weight of the plaster components. If the content of both solvents is less than the above-mentioned range, a desired effect for reducing adhesiveness may not be obtained, and if the content exceeds the above-mentioned range, a production error may occur, and thus neither is preferred.

In some embodiments, the combined content of the solvent (A) and the solvent (B) is preferably within the range of 10 to 40 wt %, and more preferably within the range of 10 to 30 wt %.

Here, the content of the solvent (A) and the solvent (B) means the content just before application to the skin. Usually, the content is kept constant from immediately after completion of the manufacturing process to just before skin application by such means as packaging with an appropriate packaging material, if necessary. Moreover, the surface of the plaster (the surface opposite to the support) is usually protected by a removal-treated solvent impermeable release film (liner).

The solvent (A) and the solvent (B) are rapidly desorbed from the plaster, particularly at the skin surface temperature (about 32° C.). Thus, while a strong adhesive force is maintained, when a patch is peeled off from the skin after use, the adhesive force of the plaster decreases and loses, and an accident due to unexpected reapplication of the adhesive can be prevented. Moreover, since these solvents are quickly desorbed even at room temperature (about 25° C.), it is possible to prevent an accident in which a child or the like accidentally applies one left uncared after opening.

### 1.1.4 Other Ingredients

In the present invention, the plaster may contain an aliphatic compound which is solid at room temperature and has a hydrophilic group (hereinafter, simply referred to as “the aliphatic compound”) as an additive, if necessary.

The aliphatic compound preferably is one having the melting point higher than room temperature, more preferably one having the melting point of 40° C. or higher, further more preferably one having the melting point of 50° C. or higher, and particularly more preferably one having the melting point of 55° C. or higher. The melting point of the aliphatic compound is preferably 50 to 80° C., more preferably 50 to 70° C., further more preferably 55 to 70° C., and particularly more preferably 55 to 65° C. Depending on the melting point, it is possible to adjust the adhesive force at the time of drug administration, and the adhesive force at the time of reapplication.

The aliphatic compound can include, but is not limited to, a C₈₋₂₂ higher alcohol, and a C₈₋₂₂ higher fatty acid. It is also possible to use a C₈₋₂₂ higher alcohol and a C₈₋₂₂ higher fatty acid in combination.

As used herein, the term “in a solid state at room temperature” means being in a solid state at about 25° C., and in a state of no fluidity at about 25° C., and a petrolatum-like semi-solid state is also included, but a solid state is preferred. Among them, those soluble in the solvent (A) and the solvent (B) are preferred.

The above-mentioned C₈₋₂₂ higher alcohol can include octyldodecanol, isostearyl alcohol, stearyl alcohol, lauryl alcohol, cetyl alcohol, palmityl alcohol, cetostearyl alcohol, decyl tetradecanol, hexyldecanol, behenyl alcohol, lauryl alcohol, and myristyl alcohol, for example.

The above-mentioned C₈₋₂₂ higher fatty acid can include lauric acid, myristic acid, palmitic acid, and stearic acid, for example.

The aliphatic compound is preferably a C₈₋₂₂ higher alcohol, more preferably a C₁₂₋₂₀ higher alcohol, further more preferably a C₁₆₋₁₈ higher alcohol, still more preferably stearyl alcohol, cetyl alcohol, or cetostearyl alcohol, and particularly preferably stearyl alcohol.

The above-mentioned aliphatic compound may be used singly or in any combination of two or more kinds. The content of the aliphatic compound is usually suitably within the range of 0.2 to 20 wt % relative to the total weight of the plaster components, preferably within the range of 0.4 to 10 wt %, and more preferably within the range of 0.4 to 5 wt %. The content of the aliphatic compound may be within the range of 0.4 to 2.5 wt %.

The plaster in the present invention may further comprise a transdermal absorption promoter and other additives, if necessary.

The transdermal absorption promoter can include fatty acids, alcohols, esters, and organic amines, for example. Specifically, the fatty acids can include caprylic acid, sorbic acid, levulinic acid, oleic acid, and the like. The alcohols can include monohydric higher alcohols such as capryl alcohol, oleyl alcohol; and multivalent alcohols such as propylene glycol, polyethylene glycol, glycerin. The esters can include propylene carbonate, diethyl sebacate, isopropyl myristate, diisopropyl adipate, myristyl palmitate, stearyl stearate, medium-chain fatty acid triglycerides. The organic amines can include monoethanolamine, monoisopropanolamine, diethanolamine, diisopropanolamine, triethanolamine, triisopropanolamine, and the like.

The above-mentioned transdermal absorption promoter may be used singly or in any combination of two or more kinds.

The additives can include conventional additives known in the art in the pharmaceutical field such as bitterants, fillers, softeners, antioxidants, pH adjusters, and perfumes. Among them, it is preferable to contain a bitterant which is effective for preventing accidental eating.

The bitterant can include denatonium and the like having a strong bitterness, for example, and denatonium benzoate is preferred. The content of the bitterant is suitably within the range of 0.005 to 0.1 wt % relative to the total weight of the plaster components, and the same applies to the case of containing denatonium benzoate. This amount may cause a strong bitter feeling when the patch is held in the mouth, and may have an effect for preventing accidental eating by a child or the like.

In the present invention, antioxidants may be comprised. The antioxidants can include water-soluble antioxidants such as sodium sulfite, sodium bisulfite, sodium hyposulfite, sodium pyrosulfite, ascorbic acid, glutathymine, and the like; and fat-soluble antioxidants such as propyl gallate, α-tocopherol. Among them, sodium sulfite and propyl gallate are preferred.

The above-mentioned antioxidants may be used singly or in any combination of two or more kinds. Moreover, it is preferable to use a water-soluble antioxidant and a fat-soluble antioxidant in combination.

The content of the antioxidants is usually suitably within the range of 0.04 to 5 wt % relative to the total weight of the plaster components, preferably within the range of 0.08 to 2 wt %, and more preferably within the range of 0.1 to 1 wt %.

### 1.2 Structure of the Present Invention Patch

The present invention patch usually comprises a plaster having fentanyl, an adhesive, the solvent (A), and the solvent (B); and a support. The plaster can be laminated on the support to form a drug layer. Therefore, in addition to the drug layer mainly composed of the plaster and the support, the present invention patch also usually has a release film (liner), and the support, the plaster, and the release film are laminated in this order.

### 1.2.1 Support in the Drug Layer

The support in the drug layer may be composed of any of a solvent-permeable material which is permeable to the solvent, and a solvent-impermeable material which is impermeable to the solvent, and is preferably composed of a solvent-impermeable material.

The solvent-permeable support material can include resins such as polyester, polypropylene, polyurethane, and vinyl chloride, a woven or nonwoven fabric composed of pulp or the like, and a porous sheet, for example Among them, a nonwoven fabric composed of polyester is preferred.

The solvent-impermeable support material can include resin films such as polyester, polyamide, and vinyl chloride, and a composite film obtained by combining a woven or nonwoven fabric with a resin film, for example. Among them, a polyester film is more preferred, and a PET (polyethylene terephthalate) film is further preferred. Among them, a nonwoven fabric laminated film in which a nonwoven fabric is laminated is preferred, and a nonwoven fabric laminated PET film is more preferred.

### 1.2.2 Present Invention Patch Having a Cover Layer

The present invention patch may be composed of only a support, a plaster, and a release film, and may further comprise a cover layer containing an adhesive layer. The cover layer is usually composed of an adhesive layer and a support. Moreover, it is preferable that the adhesive layer of the cover layer also contains a volatile organic solvent, and owing to the cover layer, the adhesive force and the ability to prevent reapplication can be increased.

It is preferable that the cover layer is larger than the plaster (drug layer) having fentanyl.

In the present invention patch having the cover layer, it is preferable that the support of the cover layer, the adhesive layer of the cover layer, the support of the drug layer, the plaster of the drug layer, and the release film are laminated in this order. Moreover, it is preferable that the release film is the same as or larger than the cover layer.

### 1.2.2.1 Adhesive Layer, Support, and Additive in the Cover Layer

The adhesive layer of the cover layer comprises an adhesive, and the adhesive usually comprises a base and a tackifier, if necessary. The base and the tackifier have the same meaning as described above.

In the present invention patch having the cover layer, the support of the plaster having fentanyl is preferably composed of a resin film, more preferably a polyester film, and further more preferably a PET film. Among them, a nonwoven fabric laminated film in which a nonwoven fabric is laminated is preferred, and a nonwoven fabric laminated PET film is more preferred.

When a nonwoven fabric laminated PET film is used as the support of the plaster having fentanyl, it is preferable that the nonwoven fabric moiety is disposed on the plaster side containing fentanyl. The plaster containing fentanyl is held on then concavo-convex moiety of the nonwoven fabric, and a drug can be applied to a predetermined site, and by bringing the flat and smooth film surface into contact with the adhesive layer of the cover layer, the adhesive force is increased, and it is possible to prevent the support from falling off.

The support of the adhesive layer of the cover layer is also preferably composed of a nonwoven fabric laminated film, and the film moiety is preferably on the adhesive layer side. Thus, by bringing the flat and smooth film surface into contact with the adhesive layer, the adhesive force is increased, and it is possible to prevent the support from falling off, and by directing the nonwoven fabric outward, the difference in feeling and appearance with the surrounding skin is reduced, and the feeling of use is excellent.

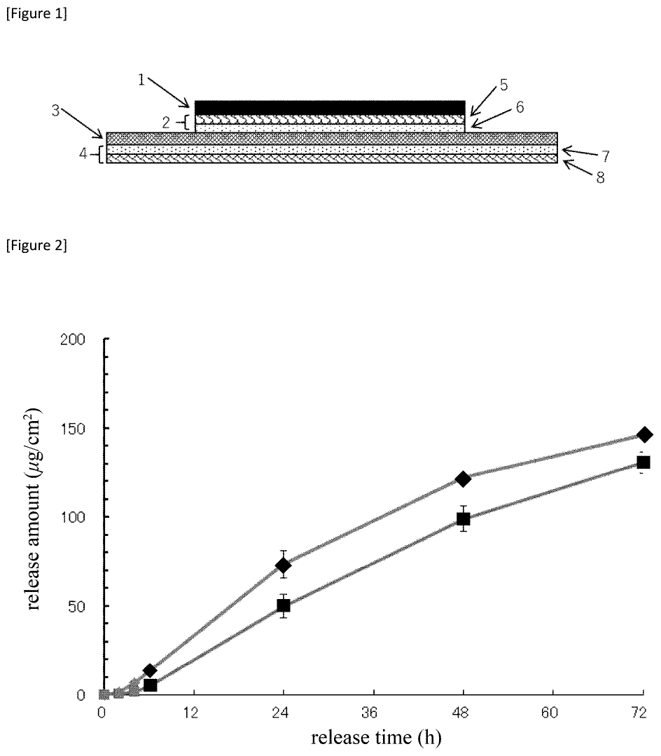

Of the configurations of the support in the present invention patch having the cover layer, a preferred embodiment is shown in FIG. 1.

Moreover, the adhesive layer of the cover layer may comprise a suitable additive, if necessary.

The additive can include conventional additives known in the art in the pharmaceutical field such as bitterants, fillers, softeners, antioxidants, pH adjusters, and perfumes. Among them, it is preferable to contain a bitterant useful for preventing accidental eating.

The bitterant can include denatonium and the like having a strong bitterness, for example, and denatonium benzoate is preferred. The content of the bitterant is suitably within the range of 0.01 to 0.1 wt % relative to the total weight of the cover layer components, and the same applies to the case of containing denatonium benzoate. This amount may cause a strong bitter feeling when the patch is held in the mouth, and may have an effect for preventing accidental eating by a child or the like.

It is also preferable that an antioxidant is comprised. The antioxidant can include water-soluble antioxidants such as sodium sulfite, sodium bisulfite, sodium hyposulfite, sodium pyrosulfite, ascorbic acid, and glutathymine; and fat-soluble antioxidants such as propyl gallate, and α-tocopherol, for example. Among them, sodium sulfite and propyl gallate are preferred.

The above-mentioned antioxidant may be used singly or in any combination of two or more kinds. Moreover, it is preferable to use a water-soluble antioxidant and a fat-soluble antioxidant in combination.

The content of the antioxidant is usually suitably within the range of 0.04 to 5 wt % relative to the total weight of the cover layer components, preferably within the range of 0.08 to 3 wt %, and more preferably within the range of 0.1 to 2 wt %.

Specific examples of other additives can include the same as those described above.

### 1.2.2.2 Volatile Organic Solvent in the Adhesive Layer of the Cover Layer

The “volatile organic solvent” in the adhesive layer of the cover layer refers to an organic solvent having the vapor pressure of 0.1 kPa or more at 20° C., and the boiling point higher than room temperature (25° C.). The volatile organic solvent is not particularly limited as long as the vapor pressure is 0.1 kPa or more at 20° C., and preferably include those having the vapor pressure of 1 kPa or more, or 1 to 50 kPa and the boiling point within the range of 65° C. to 140° C., preferably within the range of 75° C. to 125° C., for example.

Specific examples of the volatile organic solvent can include the same as those of the solvent (A) and the solvent (B) described above. Hereinafter, in this section 1.2.2.2, the solvent (A) is referred to as the solvent (C), and the solvent (B) is referred to as the solvent (D), respectively.

Alternatively, specific examples of the volatile organic solvent can include acyclic or cyclic aliphatic hydrocarbon solvents such as n-hexane, n-heptane, cyclohexane, and the like; aliphatic monoalcohol-based solvents such as ethanol, isopropyl alcohol, isobutyl alcohol, and 2-butanol; ester-based solvents such as isobutyl acetate, isopropyl acetate, and ethyl acetate; ketone-based solvents such as acetone, methyl ethyl ketone, and methyl isobutyl ketone; ether-based solvents such as diethyl ether, tetrahydrofuran, and 1,4-dioxane; and aromatic hydrocarbon solvents such as benzene and xylene.

The above-mentioned solvents may be used singly or in any combination of two or more kinds, and the solvents are preferably used in a combination of two or more kinds.

When two or more kinds of the solvents are used, it is preferable to comprise the solvent (C) and the solvent (D), and it is preferable to consist of the solvent (C) and the solvent (D).

The volatile organic solvent is preferably an acyclic or a cyclic aliphatic hydrocarbon solvent according to the solvent (C), an ester solvent according to the solvent (D), or the combination thereof. Among them, the acyclic aliphatic hydrocarbon solvent according to the solvent (C) and/or the ester solvent according to the solvent (D) is preferred, and a combination of the acyclic aliphatic hydrocarbon solvent according to the solvent (C) and the ester-based solvent according to the solvent (D) is more preferred. Specifically, ethyl acetate and/or n-heptane is preferred, and the combination of ethyl acetate and n-heptane is more preferred. When the acyclic aliphatic hydrocarbon solvent (e.g., n-heptane) according to the solvent (C) and the ester-based solvent (e.g., ethyl acetate) according to the solvent (D) are used in combination, the weight ratio thereof is preferably 1:2 to 10:1, more preferably 1:1 to 4:1, and the acyclic aliphatic hydrocarbon solvent according to the solvent (C) is preferably larger in amount than the ester solvent according to the solvent (D), and the ratio being further more preferably 1.5:1 to 3:1.

The volatile organic solvent according to the present invention is rapidly desorbed from the cover layer at skin surface temperature (about 32° C.). Thus, while a strong adhesive force is maintained, when it is peeled off from the skin after use, the adhesive force of the cover layer decreases and loses, and an accident due to unintentional reapplication of the adhesive can be prevented. Moreover, since these solvents are quickly desorbed even at room temperature (about 25° C.), it is possible to prevent an accident in which a child or the like accidentally reapply one left uncared after opening.

The content of the volatile organic solvent according to the present invention is suitably within the range of 2 to 35 wt % relative to the total weight of the cover layer components. If the content of the solvent is less than the above-mentioned range, a desired effect for reducing adhesiveness may not be obtained, and if the content exceeds the above-mentioned range, a production error may occur, and thus neither is preferable.

In some embodiments, the content of the solvent is preferably from 3 to 20 wt %, more preferably from 5 to 10 wt %.

### 1.2.3 Release Film

The release film according to the present invention mainly protects the plaster (drug layer) surface and the cover layer surface before the use of the present invention patch, and also suppresses volatilization of the solvent (A), the solvent (B), and the volatile organic solvent before use.

The release film is not particularly limited as long as it has the above-mentioned function, and is usually a paper, a film made of plastic, a sheet made of plastic, or a laminated film. Specific examples of the release film can include polyester resins such as polyethylene terephthalate (PET) and polybutylene terephthalate, polyolefin resins such as polyethylene and polypropylene, and may be a laminated film obtained by laminating these resins with metals such as aluminum or other resins. When paper is used for the release film, treatment with some release agent is usually necessary. Such a release agent can include silicone, for example, and siliconized paper treated with a silicone release agent, or the like is preferably used.

Among the above-mentioned, the release film made of a polyester resin is preferred, and the release film made of PET is more preferred. Moreover, it is preferable that the silicone treatment is performed in order to facilitate peeling.

The release film may be one sheet, or a plurality of sheets which is superimposed while usually being shifted. Moreover, it is preferable to have a cut in the release film to be easily peeled off at the time of use.

### 1.3 Method for Producing the Present Invention Patch

The present invention patch can be produced, for example, by mixing fentanyl, an adhesive, the solvent (A), the solvent (B), and the like, obtaining a mixture, coating the mixture on a release film or a support, drying, and sticking the support or the release film to the obtained plaster surface. Preferred production methods are shown below.

The solvent (A) and the solvent (B) are added in amounts of more than the prescription amounts (required amounts), and fentanyl, an adhesive and, if necessary, an additive, are mixed to produce a coating liquid, and then the coating liquid is coated on a release film. Upon drying the excess portion of the excessively added solvent is removed by evaporation to obtain the plaster, and then the support is laminated onto the plaster.

Thus, a patch is obtained wherein the plaster contains fentanyl and an adhesive, and further contains a predetermined amount of the solvent (A) and the solvent (B). Preferably, a patch which contains the solvent (A) and the solvent (B) within the range of 5 to 45 wt %, is obtained.

The laminated present invention patch, which is a more preferred embodiment of the present invention, can be produced in the same manner. Specifically, a volatile organic solvent is added to a larger amount than a prescription amount (required amount), and an adhesive and, if necessary, an additive are mixed to produce a coating liquid, and then the coating liquid is coated on a release film. Upon drying, the excess portion of the excessively added solvent is removed by evaporation to obtain an adhesive layer, and a support is laminated on the adhesive layer. Next, the release film on the adhesive layer is peeled off, and the fentanyl-containing patch (drug layer) obtained by the above method is adhered, and the release film is adhered again to obtain a laminated patch. Prior to sticking the release film, the release film on the plaster may be peeled off, if necessary.

In the present invention, it is preferable to have a structure in which fentanyl is not contained in the adhesive layer of the cover layer, and further, the content of the solvent (A) and the solvent (B) in the plaster is suitably set within the range of 5 to 45 wt % relative to the total weight of the plaster components, but as for the volatile organic solvent in the adhesive layer of the cover layer, the content is suitably within the range of 2 to 35 wt % relative to the total weight of the adhesive layer components of the cover layer. The above-mentioned amount is preferred.

The thus obtained patch is bonded to obtain the laminated present invention patch. The obtained present invention patch can be packaged in an appropriate package. The package can include a bag made of a film, for example, and a multilayer film is preferred as the film, and an aluminum laminated film is more preferred, and polyethylene and PET are preferred as the innermost layer thereof.

Here, the excess amounts of the solvents added when the solvent (A) and the solvent (B), or two or more kinds of volatile organic solvents are used, may be dried by adding excess amounts of two or more kinds of the solvents in a ratio corresponding to the vapor pressure at the time of drying or evaporation step, and the drying step may be terminated when the excess amount of the solvents reaches a predetermined weight to adjust the weight of the solvents. For more accuracy, the solvent in the obtained patch may be quantified by gas chromatography or liquid chromatography to adjust the excess amount thereof.

### EXAMPLE

Hereinafter, the present invention will be illustrated in more detail with reference to Examples, but the present invention is not limited to the following.

### [Evaluation Method]

### (Evaluation 1) Reapplication Adhesiveness Test

From the consideration that fentanyl in the prescription amount in the Examples does not substantially affect the adhesiveness of the patch, or other thoughts, each preparation was produced in such a way that fentanyl was not contained but other components were mixed at the ratios shown in the table. The release film was peeled off to expose the plaster, and the preparation was left at room temperature for 30 minutes, and the plaster of each preparation at 0 minutes and 30 minutes after the start of standing, was touched by hand to confirm the adhesive force, and then applied to a forearm portion to evaluate the reapplication adhesiveness of each preparation.

### (Evaluation 2) Sustained Release Test

The test preparation was applied to a pig skin placed in a Franz type diffusion cell, and the cumulative permeation amount of fentanyl that had permeated through the skin, was measured at each sampling point, and the ratio of the non-permeated amount (residual ratio) at each sampling point was calculated by the following formula. Sampling points were at 24 hours, 48 hours, and 72 hours after the start of application.

Residual ratio (%)=(the amount of fentanyl in a plaster at initial time−the cumulative permeation amount of fentanyl)÷(the amount of fentanyl in a plaster at initial time)×100 [Equation 1]

The larger the above ratio is, the more excellent the sustained release property is, in general.

### (Evaluation 3) Reapplication Skin Permeability Test

The release liner of the test preparation was peeled off, and at room temperature, one side of the plaster was exposed to air for 30 minutes to desorb ethyl acetate and heptane from the plaster. Subsequently, the skin permeability of fentanyl in the test preparation was evaluated by applying the test preparation to a pig skin placed in a Franz type diffusion cell and measuring the cumulative permeation amounts of fentanyl that had permeated through the skin until 24 hours and 48 hours after application of the test preparation.

### Example 1 and Comparative Example 1

The patches of Example 1 and Comparative Example 1 having the composition shown in Table 1 were produced in the following manner. The composition shown in Table 1 was weighed, and ethyl acetate and heptane were added to a larger amount than in the prescription and mixed to produce a coating liquid. The coating liquid was coated on a siliconized PET film (release film) and dried, and an excess portion of the solvent excessively added was evaporated, and the PET film side of the nonwoven fabric laminated PET film (support) was bonded onto the plaster and laminated at 70° C., thereby obtaining the respective patches of Example 1 and Comparative Example 1. The resulting patch was placed in an aluminum laminated bag and sealed.

For each of the patches of Example 1 and Comparative Example 1, the reapplication adhesiveness test (Evaluation 1) and the sustained release test (Evaluation 2) were performed to obtain the results shown in Table 1, respectively.

TABLE 1

| | | | | Comparative |
|---|---|---|---|---|
| | | | Example 1 | Example 1 |
| Plaster | Drug | Fentanyl | 6.67 | 7.16 |
| | Adhesive | Styrene-Isoprene-Styrene | 45.89 | 49.21 |
| | | Copolymer | | |
| | | Terpene Resin | 9.18 | 9.84 |
| | | Polyisobutylene | 5.51 | 5.91 |
| | Solvent (A) | Heptane | 18.86 | 8.05 |
| | Solvent (B) | Ethyl Acetate | 13.77 | 19.69 |
| | Additive | Sodium Bisulfite | 0.08 | 0.09 |
| | | Propyl Gallate | 0.04 | 0.04 |
| | | Denatonium Benzoate | 0.01 | 0.01 |
| | (wt %) | Total | 100.00 | 100.00 |
| | Evaluation 1 | Immediately after opening | Adhesion | Adhesion |
| | | After leaving for 30 min. | No adhesion | No adhesion |
| | Evaluation 2 | 0 hours | 100 | 100 |
| | Residual amount of | 24 hours | 91 | 83 |
| | fentanyl (%) | 48 hours | 83 | 69 |
| | | 72 hours | 76 | 61 |

As shown in Table 1, in any of the preparations of Example 1 and Comparative Example 1, the adhesive force was felt at 0 minutes after being left standing (immediately after opening), and then it did not fall off even when it was applied to the forearm portion. In addition, 30 minutes after leaving, neither of the preparations showed adhesiveness and could not be applied to the forearm. On the other hand, in Example 1, since the residual amount of fentanyl was larger than that of Comparative Example 1, fentanyl gradually permeated through the skin, and as for sustained release, the present invention patch according to Example 1 was excellent.

### Example 2

The present invention patch of Example 2 having the composition shown in Table 2 was produced in the following manner.

The compositions shown in the sections of “Plaster of the drug layer” and “Adhesive layer of the cover layer” of Table 2 were respectively weighed, and ethyl acetate and heptane were added to a larger amount than the prescription and mixed to produce each coating liquid. Each coating liquid was coated on a siliconized PET film (release film) and dried, and an excess portion of the solvent excessively added was evaporated, so that a nonwoven fabric laminated PET film (a support of a drug layer and a cover layer) was bonded on a plaster and laminated at 70° C., thereby producing a drug layer comprising a plaster and a support, and a cover layer of Example 2. At this time, the support of the drug layer was bonded onto the plaster by the nonwoven fabric side, and the support of the cover layer was bonded onto the adhesive layer by the PET film side.

Further, the release film of the cover layer was removed, the cover layer was adhered to the drug layer so that the support of the drug layer was in contact with the surface of the adhesive layer, the release film on the plaster side of the drug layer was removed, and then the siliconized PET film (release film) was laminated so as to cover the plaster and the adhesive layer of the cover layer, to obtain the present invention patch having a laminated structure. The obtained present invention patch was placed in an aluminum laminate bag and sealed.

Note that DURO-TAK 387-2287 (trade name, manufactured by Henkel Japan) was used as the acrylic polymer. The same applies to the other examples.

TABLE 2

| | | | Example 2 |
|---|---|---|---|
| Plaster of | Drug | Fentanyl | 6.6 |
| Drug layer | Adhesive | Styrene-Isoprene-Styrene | 45.34 |
| | | Copolymer | |
| | | Terpene Resin | 9.07 |
| | | Acrylic Polymer | 19.8 |
| | Solvent (A) | Heptane | 11.72 |
| | Solvent (B) | Ethyl Acetate | 5.81 |
| | Additive | Stearyl Alcohol | 1.65 |
| | | Denatonium Benzoate | 0.01 |
| | (wt %) | Total | 100.00 |
| Support of | Nonwoven fabric laminated PET film | | |
| Drug layer | | | |
| Adhesive of | Adhesive | Styrene-Isoprene-Styrene | 16.92 |
| Cover layer | | Copolymer | |
| | | Terpene Resin | 34.57 |
| | | Acrylic Polymer | 36.77 |
| | Solvent | Ethyl Acetate | 3.00 |
| | | Heptane | 6.00 |
| | | Water | 0.37 |
| | Additive | Propyl Gallate | 0.05 |
| | | Denatonium Benzoate | 0.01 |
| | | Sodium Pyrosulfite | 0.10 |
| | | Stearyl Alcohol | 2.21 |
| | (wt %) | Total | 100.00 |

| | |
|---|---|
| Support of | Nonwoven fabric laminated PET film |
| Cover layer | |

| | | |
|---|---|---|
| Evaluation 1 | Immediately after opening | Adhesion |
| | After leaving for 30 min. | No adhesion |
| Evaluation 3 | Immediately after opening | 112 |
| (μg/cm², 24 hours) | After leaving for 30 min. | 49 |

As shown in Table 2, the present invention patch of Example 2 exhibited adhesive force at 0 minutes of standing (immediately after opening), and then did not fall off when it was applied to a forearm portion. After being left standing for 30 minutes, the adhesive force was not felt, and it was not possible to apply it to the forearm. Then, the cumulative amount of skin permeation for 24 hours was remarkably smaller after exposure to air for 30 minutes as compared to that at 0 minutes of standing (immediately after opening).

### Example 3 to 6

In the same manner as in Example 1, each of the patches shown in Table 3 below was obtained. The resulting patch was placed in an aluminum laminate bag and sealed.

TABLE 3

| | | | Ex. 3 | Ex. 4 | Ex. 5 | Ex. 6 |
|---|---|---|---|---|---|---|
| Plaster | Drug | Fentanyl | 2.00 | 1.82 | 1.92 | 2.00 |
| | Adhesive | Styrene-Isoprene- | 25.00 | 22.77 | 23.95 | 25.00 |
| | | Styrene Copolymer | | | | |
| | | Terpene Resin | 17.00 | 15.48 | 16.28 | 17.00 |
| | | Acrylic Polymer | 33.50 | 30.97 | 32.57 | 34.00 |
| | Solvent (A) | Heptane | 16 | 21.1 | 18.6 | 16 |
| | Solvent (B) | Ethyl Acetate | 5.50 | 7.4 | 6.2 | 5.5 |
| | Additive | Stearyl Alcohol | 1.00 | 0.46 | 0.48 | 0.5 |
| | (wt %) | Total | 100.00 | 100.00 | 100.00 | 100.00 |

Reapplication skin permeability test (Evaluation 3) was performed on the patch of Example 4 to obtain the results shown in Table 4, respectively.

TABLE 4

| Time (hrs) | 0 | 2 | 4 | 6 | 24 | 48 |
|---|---|---|---|---|---|---|
| Immediately after opening | 0 | 0.68 | 4.19 | 11.27 | 68.97 | 118.99 |
| After leaving for 30 minutes | 0 | 0 | 0 | 0.22 | 11.87 | 35.26 |

As shown in Table 4, in Example 4, the cumulative skin permeation amount for 48 hours was remarkably smaller after exposure to air for 30 minutes as compared to that at 0 minutes of standing (immediately after opening). Moreover, in the present invention patch of Example 4, the release of fentanyl was continued for a long time from immediately after opening and sustained.

### [Test Example 1] Comparison with Commercial Products

(1) The release liner of the present invention patch of Example 5 and the commercially available fentanyl transdermal absorption type preparation, was peeled off, and each preparation was applied to a pig skin placed in the Franz type diffusion cell, and the amount of transdermal permeation (release amount) of fentanyl from each preparation was measured by measuring the amount of permeation of fentanyl that had permeated through the skin. The results are shown in FIG. 2.

As shown in FIG. 2, both preparations showed the similar release pattern of fentanyl, and the both release patterns were also of sustained release.

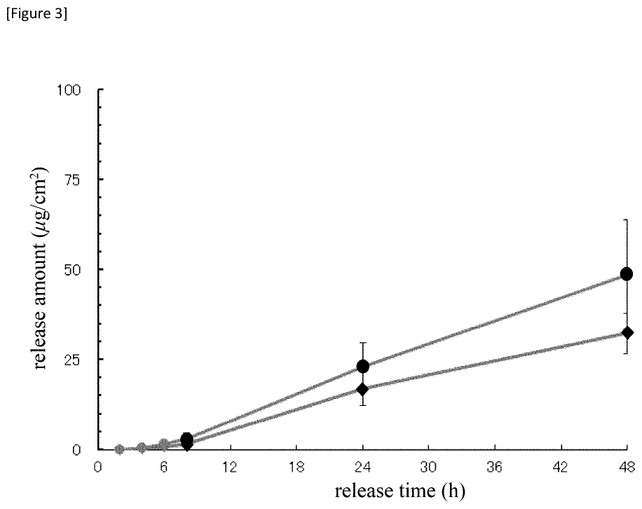

(2) Each preparation was applied to a pig skin placed in the Franz type diffusion cell in the same manner as described above, and after 24 hours, each preparation was peeled off from the pig skin, left for 60 minutes, and then reapplied to a new pig skin to measure the amount of skin permeation (release amount) of fentanyl from each preparation until 48 hours. The result is shown in FIG. 3.

As shown in FIG. 2, the skin permeability immediately after opening did not differ greatly between the two, but after reapplication, the skin permeability of the present invention patch decreased more than that of the commercial product as shown in FIG. 3. From this, it has been suggested that, for example, even if a patient peels off a preparation after use and leaves it, and the preparation being accidentally stuck to a child or the like, since the skin permeability is lowered in the present invention patch, so that it is excellent in preventing an accident and safer.

### [Test Example 2] Concentration Change of Solvent

Concentrations of heptane (solvent (A)) and ethyl acetate (solvent (B)) of Example 4 at the time of opening the present invention patch and after standing for 30 minutes after opening were measured (n=2). The results are shown in Table 5 below.

TABLE 5

| | Conc. of Heptane | Conc. of Ethyl Acetate |
|---|---|---|
| | (w/w %) | (w/w %) |
| Immediately after opening | 21.1 | 7.4 |
| After leaving for 30 minutes | 2.5 | 0.4 |

As shown in Table 5, when left for 30 minutes after opening, the concentration of heptane and ethyl acetate in the present invention patch was extremely reduced. It was suggested that this contributes to the decrease in the adhesive force of the present invention patch and the decrease in transdermal absorbability after the opening.

### [Test Example 3] Adhesion Evaluation

Since it is considered that the prescription amount of fentanyl in the Examples does not substantially affect the adhesive force of the patch, the adhesive force was evaluated for the placebo of the present invention patch containing no fentanyl. Specific prescriptions are shown in Table 6 below.

TABLE 6

| | | | Placebo 1 | Placebo 2 |
|---|---|---|---|---|
| Plaster | Drug | Fentanyl | — | — |
| | Adhesive | Styrene-Isoprene- | 25.70 | 24.58 |
| | | Styrene Copolymer | | |
| | | Terpene Resin | 13.77 | 19.18 |
| | | Acrylic Polymer | 31.21 | 32.45 |
| | Solvent (A) | Heptane | 21.2 | 18.4 |
| | Solvent (B) | Ethyl Acetate | 7.2 | 4.9 |
| | Additive | Stearyl Alcohol | 0.92 | 0.49 |
| | (wt %) | Total | 100.00 | 100.00 |

The test was conducted according to the rolling ball tack test method described in the section of Adhesion Test Method (General Test Method 6.13) of the 17th amendment Japanese Pharmacopoeia (n=5). The results are shown in Table 7 below.

TABLE 7

| | | Moving distance (mm) | | | |
|---|---|---|---|---|---|
| | | Leave for | Leave for | Leave for | Leave for |
| | | 0 min. | 5 min. | 10 min. | 30 min. |
| Placebo 1 | Average | 21.4 | 30.4 | 30.6 | 31.2 |
| | Standard deviation | 1.1 | 2.4 | 3.4 | 3.0 |
| | %, Leave for 0 min. | — | 145 | 146 | 149 |
| Placebo 2 | Average | 22.6 | 26.4 | 29.4 | 49.8 |
| | Standard deviation | 2.7 | 1.5 | 2.5 | 12.8 |
| | %, Leave for 0 min. | — | 120 | 134 | 226 |

As shown in Table 7, it is evident that the placebo 1 and 2 of the present invention patch have significantly reduced adhesive force from about 5 minutes after being left open.

### INDUSTRIAL APPLICABILITY

Since the present invention patch can suppress reapplication after use and also reduce dermal absorbability at the time of reapplication, it is possible to effectively prevent the risk due to an abuse of drugs and an accident involving drugs. In addition, it is also advantageous in the adjustment of skin absorption or pharmacokinetic profile of fentanyl. Therefore, it is useful as a long-time usable patch excellent in sustained release of fentanyl.

1. A patch containing fentanyl as an active ingredient,
comprising a plaster having fentanyl, an adhesive, a volatile hydrocarbon solvent (A), and a heteroatom-containing volatile organic solvent (B), wherein the boiling point of the solvent (A) is higher than that of the solvent (B), and the content of the solvent (A) in the plaster is larger than that of the solvent (B) in the plaster.

2. The patch according to claim 1, wherein the boiling point of the solvent (B) is lower than that of the solvent (A) by 8° C. or more.

3. The patch according to claim 1, wherein the ratio of the solvent (B) content in the plaster to the solvent (A) content in the plaster is within the range of 1:1.1 to 1:20 (B:A) by weight.

4. The patch according to claim 1, wherein the boiling point of the solvent (A) is within the range of 75 to 140° C.

5. The patch according to claim 1, wherein the content of the solvent (B) in the plaster is within the range of 1 to 30 wt %.

6. The patch according to claim 1, wherein the solvent (B) is one or more solvents selected from the group consisting of an ester-based solvent, an ether-based solvent, an alcohol-based solvent, an amine-based solvent, a ketone-based solvent, an amide-based solvent, and a chloride-based solvent.

7. The patch according to claim 1, wherein the solvent (B) is one or more solvents selected from the group consisting of ethyl acetate, ethanol, and isopropanol.

8. The patch according to claim 1, wherein the solvent (A) is one or more solvents selected from the group consisting of n-heptane, toluene, cyclohexane, and n-octane.

9. The patch according to claim 1, wherein the adhesive is a mixture of an acrylic polymer and a styrene-isoprene-styrene copolymer.

10. The patch according to claim 9, wherein the weight ratio of the acrylic polymer to the styrene-isoprene-styrene copolymer in the mixture is within the range of 5:1 to 1:5.

11. The patch according to claim 1, further comprising a bitterant.

12. The patch according to claim 1, further comprising a C₈₋₂₂ higher alcohol.

13. A patch containing fentanyl as an active ingredient,
comprising a drug layer consisting of a plaster having fentanyl, an adhesive, a volatile hydrocarbon solvent (A), and a heteroatom-containing volatile organic solvent (B), and a support; and a cover layer covering and larger than the drug layer, wherein the cover layer comprises an adhesive layer; and wherein the boiling point of the solvent (A) is higher than that of the solvent (B) and the content of the solvent (A) in the plaster is larger than that of the solvent (B) in the plaster.

14. The patch according to claim 13, wherein the adhesive layer contains a volatile organic solvent.

15. The patch according to claim 14, wherein the volatile organic solvent in the adhesive layer is a volatile hydrocarbon solvent (C), a heteroatom-containing volatile organic solvent (D), or a mixed solvent thereof.

16. The patch according to claim 15, wherein the solvent (C) is an acyclic or a cyclic aliphatic hydrocarbon solvent, and the solvent (D) is one or more solvents selected from the group consisting of an ester-based solvent, an ether-based solvent, an alcohol-based solvent, an amine-based solvent, a ketone-based solvent, an amide-based solvent, and a chloride-based solvent.

17. The patch according to claim 15 or 16, wherein the solvent (C) is one or more solvents selected from the group consisting of n-heptane, toluene, cyclohexane, and n-octane, and the solvent (D) is one or more solvents selected from the group consisting of ethyl acetate, ethanol, and isopropanol.

18. The patch according to claim 15, wherein the solvent (C) is n-heptane and the solvent (D) is ethyl acetate.

19. The patch according to claim 12, wherein the C₈₋₂₂ higher alcohol is C₈₋₂₂ higher alcohol is stearyl alcohol.
